(19) United States
(12) Patent Application Publication
(10) Pub. No.: US 2026/0054084 A1
(43) Pub. Date: Feb. 26, 2026

(54) TRANSCRANIAL MAGNETIC STIMULATION COIL

(71) Applicant: INSTITUTE OF BIOMEDICAL ENGINEERING, CHINESE ACADEMY OF MEDICAL SCIENCES, Tianjin (CN)

(72) Inventors: Tao YIN, Tianjin (CN); Xin WANG, Tianjin (CN); Zhipeng LIU, Tianjin (CN); Ren MA, Tianjin (CN); Jingna JIN, Tianjin (CN); He WANG, Tianjin (CN); Ying LI, Tianjin (CN); Jiankang WU, Tianjin (CN); Shunqi ZHANG, Tianjin (CN); Xiaoqing ZHOU, Tianjin (CN)

(21) Appl. No.: 19/099,010

(22) PCT Filed: Oct. 11, 2023
(86) PCT No.: PCT/CN2023/123944

§ 371 Date: Jan. 27, 2025

(30) Foreign Application Priority Data
May 15, 2023 (CN) 202310544528.4

(51) Int. Cl. A61N 2/02; A61N 2/00

(52) U.S. Cl. CPC A61N 2/02; A61N 2/006

(57) ABSTRACT

Provided is a transcranial magnetic stimulation coil. The transcranial magnetic stimulation coil includes n stereoscopic units. A plane where each of the n stereoscopic units is located intersects a plane where a to-be-stimulated target region is located. Each of the n stereoscopic units includes a start point and an end point, and an end point of an i^{th} stereoscopic unit among the n stereoscopic units is connected to a start point of an (i+1)^{th} stereoscopic unit among the n stereoscopic units. The transcranial magnetic stimulation coil is arranged in a spiral shape, and for any one of the n stereoscopic units on a side of the transcranial magnetic stimulation coil facing an edge of the to-be-stimulated target region, a minimum distance between a projection shape within the to-be-stimulated target region and the edge of the to-be-stimulated target region is less than a first preset distance.

### CROSS-REFERENCE TO RELATED APPLICATIONS

This is a National Stage Application, filed under 35 U.S. C. 371, of International Patent Application No. PCT/CN2023/123944, filed on Oct. 11, 2023, which claims priority to Chinese Patent Application No. 202310544528.4 filed with the China National Intellectual Property Administration (CNIPA) on May 15, 2023, the disclosures of which are incorporated herein by reference in their entireties.

### TECHNICAL FIELD

The present application relates to the field of transcranial magnetic stimulation technology, for example, a transcranial magnetic stimulation coil.

### BACKGROUND

Transcranial magnetic stimulation technology is a non-invasive brain regulation method. A magnetic signal can pass through a skull without attenuation to stimulate brain nerves. A practical application is not limited to brain stimulation. Peripheral nerves and muscles can also be stimulated. With the development of technology, transcranial magnetic stimulation technology has gained increasing recognition in the fields of clinical mental diseases, neurological diseases and rehabilitation.

In the related art, transcranial magnetic stimulation coils are mainly ring coils and figure-eight coils. A stimulation region of the ring coil is a ring-shaped region below the coil. The figure-eight coil consists of two ring coils, and a stimulation region of the figure-eight coil is below the position where the two rings overlap. The transcranial magnetic stimulation coils in the related art have the problem that the stimulation region of the coil cannot be completely matched with a to-be-stimulated brain function target region.

### SUMMARY

Embodiments of the present application provide a transcranial magnetic stimulation coil to match a transcranial magnetic stimulation region with a to-be-stimulated target region and improve stimulation accuracy.

The transcranial magnetic stimulation coil provided in the embodiments of the present application includes n stereoscopic units. A plane where each stereoscopic unit is located intersects a plane where a to-be-stimulated target region is located.

Each stereoscopic unit includes a start point and an end point, and the end point of an i^{th} stereoscopic unit among the n stereoscopic units is connected to the start point of an (i+1)^{th} stereoscopic unit among the n stereoscopic units, where 1≤i≤n−1, and both i and n are positive integers.

The transcranial magnetic stimulation coil is arranged in a spiral shape, and for any one of the n stereoscopic units on a side of the transcranial magnetic stimulation coil facing an edge of the to-be-stimulated target region, the minimum distance between a projection shape within the to-be-stimulated target region and the edge of the to-be-stimulated target region is less than a first preset distance.

### BRIEF DESCRIPTION OF DRAWINGS

FIG. 1 is a schematic diagram of a projection of a transcranial magnetic stimulation coil within a to-be-stimulated target region according to an embodiment of the present application.

FIG. 2 is a structure diagram of a stereoscopic unit according to an embodiment of the present application.

FIG. 3 is a structure diagram of another stereoscopic unit according to an embodiment of the present application.

FIG. 4 is a structure diagram of another stereoscopic unit according to an embodiment of the present application.

FIG. 5 is a structure diagram of an n^{th} stereoscopic unit according to an embodiment of the present application.

FIG. 6 is a schematic diagram of a projection of another transcranial magnetic stimulation coil within a to-be-stimulated target region according to an embodiment of the present application.

### REFERENCE LIST

101 stereoscopic unit
20 to-be-stimulated target region
10 transcranial magnetic stimulation coil
11 start point
12 end point
1011 first bottom segment
1012 first rising segment
1013 first top segment
1014 first falling segment
30 second bottom segment
40 second rising segment
50 housing

### DETAILED DESCRIPTION

The present application is described below in conjunction with drawings and embodiments. It is to be understood that the embodiments described herein are intended to illustrate the present application Additionally, it is to be noted that for ease of description, only part, not all, of structures related to the present application are illustrated in the drawings.

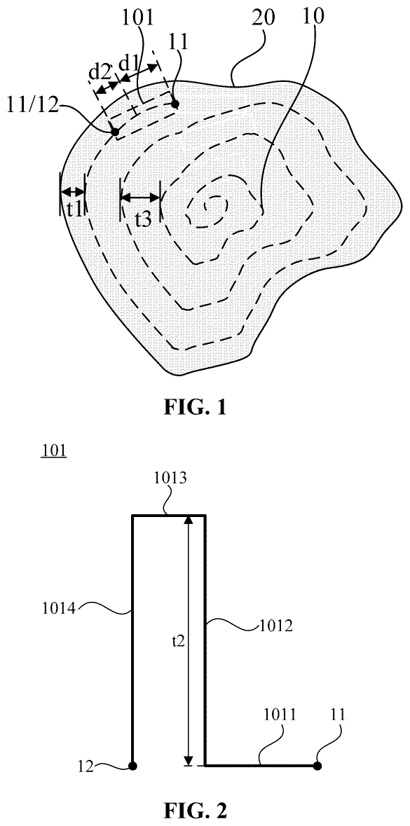

FIG. 1 is a schematic diagram of a projection of a transcranial magnetic stimulation coil 10 within a to-be-stimulated target region 20 according to an embodiment of the present application. As shown in FIG. 1, the transcranial magnetic stimulation coil 10 includes n stereoscopic units 101. A plane where each stereoscopic unit 101 is located intersects a plane where a to-be-stimulated target region 20 is located. Each stereoscopic unit 101 includes a start point 11 and an end point 12, and the end point 12 of an i^{th} stereoscopic unit 101 among the n stereoscopic units 101 is connected to the start point 11 of an (i+1)^{th} stereoscopic unit 101 among the n stereoscopic units 101, where 1≤i≤n−1, and both i and n are positive integers. The transcranial magnetic stimulation coil 10 is arranged in a spiral shape, and for any one of the n stereoscopic units 101 on a side of the transcranial magnetic stimulation coil 10 facing an edge of the to-be-stimulated target region 20, the minimum distance t1 between a projection shape within the to-be-stimulated target region 20 and the edge of the to-be-stimulated target region 20 is less than a first preset distance.

For example, the transcranial magnetic stimulation coil 10 includes n stereoscopic units 101. The plane where each stereoscopic unit 101 is located intersects the plane where the to-be-stimulated target region 20 is located. That is, a plane may be formed by each stereoscopic unit 101 and intersects the plane where the to-be-stimulated target region 20 is located. An included angle between planes formed by two adjacent stereoscopic units 101 may be determined according to a shape of the edge of the to-be-stimulated target region 20.

Each stereoscopic unit 101 includes the start point 11 and the end point 12, and the end point 12 of the i^{th} stereoscopic unit 101 is connected to the start point 11 of the (i+1)^{th} stereoscopic unit 101. That is, the transcranial magnetic stimulation coil 10 is formed by winding multiple stereoscopic units 101 along the to-be-stimulated target region 20. The multiple stereoscopic units 101 are connected end to end and may be continuously arranged in the to-be-stimulated target region 20, that is, local stimulation or overall stimulation of the to-be-stimulated target region 20 can be achieved.

In an embodiment, the transcranial magnetic stimulation coil 10 is arranged in a spiral shape. A winding region of the transcranial magnetic stimulation coil 10 may be determined according to the shape of the to-be-stimulated target region 20. The winding starts from the edge of the to-be-stimulated target region 20, the stereoscopic unit 101 is folded inward after the winding is performed for one cycle, and the winding continues to be performed until the entire to-be-stimulated target region 20 is filled so that a coil matching with the shape of the to-be-stimulated target region 20 is formed. In the winding process of the transcranial magnetic stimulation coil 10, a connection angle between two adjacent stereoscopic units 101 is determined by an arc of the edge of the to-be-stimulated target region 20 which is located at a position where the two adjacent stereoscopic units 101 are connected; thus, a winded coil that fits the shape of the to-be-stimulated target region 20 can be formed and completely cover the entire to-be-stimulated target region 20, that is, the overall stimulation can be performed on the to-be-stimulated target region 20.

In an embodiment, for any one of the n stereoscopic units 101 on the side of the transcranial magnetic stimulation coil 10 facing the edge of the to-be-stimulated target region 20, the minimum distance t1 between the projection shape of the stereoscopic unit 101 within the to-be-stimulated target region 20 and the edge of the to-be-stimulated target region 20 is less than the first preset distance. The minimum distance t1 may be understood as the minimum linear distance between the projection shape of any one of the stereoscopic units 101 within the to-be-stimulated target region 20 and the edge of the to-be-stimulated target region 20. The first preset distance may be understood as a fixed distance value between the projection shape of the stereoscopic unit 101 within the to-be-stimulated target region 20 and the edge of the to-be-stimulated target region 20. For example, the first preset distance may be 1 centimeter (cm), 3 cm, or 5 cm. That is, for any one of the stereoscopic units 101 on the side of the transcranial magnetic stimulation coil 10 near the edge of the to-be-stimulated target region 20, the minimum distance t1 between the projection shape within the to-be-stimulated target region 20 and the edge of the to-be-stimulated target region 20 is less than the fixed distance value. That is, a connection angle between the start point 11 and the end point 12 of two adjacent stereoscopic units 101 matches the arc of the edge of the to-be-stimulated target region 20. That is, the projection shape of the stereoscopic unit 101 located at the edge of the to-be-stimulated target region 20 within the to-be-stimulated target region 20 is the same as the shape of the edge of the to-be-stimulated target region 20, so that a transcranial magnetic stimulation region can match with a brain function target region, thereby improving stimulation accuracy.

For example, the to-be-stimulated target region 20 may be a local region of the brain function region or the entire region of the brain function region. Since the shape of the to-be-stimulated target region 20 may be irregular and an angle less than 90° may exist at the edge, the bending degree of the coil is relatively large when the coil is winded along the to-be-stimulated target region 20, it is prone to abrasion or breakage. A relatively hard coil may not fit the shape of the to-be-stimulated target region 20.

As a comparative embodiment, the design of a ring coil, a figure-eight coil or other types of transcranial magnetic stimulation coils used in the related art mainly considers stimulation intensity, stimulation depth and stimulation focusing and does not consider the matching between the stimulation shape and the brain function target region. When the stimulation target region is a portion of the brain function region, the focusing region of the magnetic stimulation coil is generally aligned to the to-be-stimulated target region 20 to achieve focused stimulation. When the stimulation target region is the entire brain function region, this type of coil can only stimulate a portion of the brain function region and does not completely stimulate the brain function region, or may cover a region beyond the brain function region, resulting in unnecessary stimulation to the region beyond the brain function region.

The transcranial magnetic stimulation coil 10 provided in the embodiment of the present application includes the n stereoscopic units 101. The plane where each stereoscopic unit 101 is located intersects the plane where the to-be-stimulated target region 20 is located and can perform magnetic stimulation on the to-be-stimulated target region 20. In addition, the end point 12 of the i^{th} stereoscopic unit 101 among the n stereoscopic units 101 is connected to the start point 11 of the (i+1)^{th} stereoscopic unit 101 among the n stereoscopic units 101. Each stereoscopic unit 101 can generate a corresponding stimulation point, and stimulation points of multiple stereoscopic units 101 form a lattice that contributes to generation of an irregularly shaped stimulation region. That is, the transcranial magnetic stimulation coil 10 formed by the multiple stereoscopic units 101 can be continuously arranged in the to-be-stimulated target region 20. That is, the local stimulation or the overall stimulation of the to-be-stimulated target region 20 can be performed. The transcranial magnetic stimulation coil 10 is arranged in a spiral shape, and for any one of the n stereoscopic units 101 on the side of the transcranial magnetic stimulation coil 10 facing the edge of the to-be-stimulated target region 20, the minimum distance t1 between the projection shape of the stereoscopic unit 101 within the to-be-stimulated target region 20 and the edge of the to-be-stimulated target region 20 is less than the first preset distance. That is, the projection shape of the stereoscopic unit 101 located at the edge of the to-be-stimulated target region 20 within the to-be-stimulated target region 20 is the same as the shape of the edge of the to-be-stimulated target region 20 so that the matching between the transcranial magnetic stimulation region and the brain function target region can be implemented, thereby improving the stimulation accuracy.

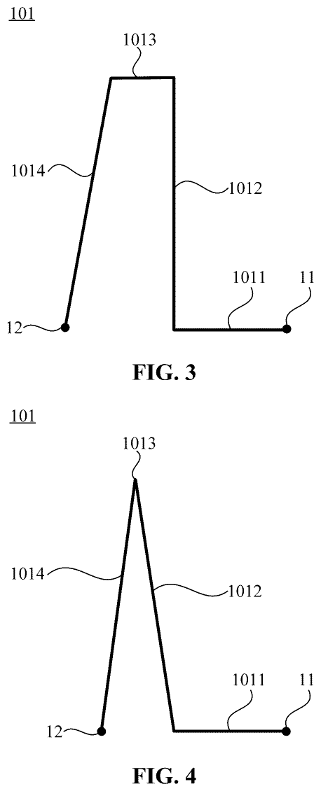

FIG. 2 is a structure diagram of a stereoscopic unit 101 according to an embodiment of the present application. FIG. 3 is a structure diagram of another stereoscopic unit 101 according to an embodiment of the present application. As shown in FIGS. 2 and 3, at least some of the n stereoscopic units 101 include a first bottom segment 1011, a first rising segment 1012, a first top segment 1013 and a first falling segment 1014 that are connected in sequence, an end of the first bottom segment 1011 includes the start point 11, and an end of the first falling segment 1014 includes the end point 12.

For example, the shape of the to-be-stimulated target region 20 is determined to be irregular according to a brain atlas, a coil winding region is determined according to the shape of the to-be-stimulated target region 20, and the end point 12 of the first falling segment 1014 of each stereoscopic unit 101 is connected to the start point 11 of the first bottom segment 1011 of the next stereoscopic unit 101. In this manner, a connection angle between two adjacent stereoscopic units 101 can be determined by the arc of the edge of the to-be-stimulated target region 20 located at the position of the connection angle, and multiple stereoscopic units 101 connected in sequence can form a transcranial magnetic stimulation coil 10 that fits the shape of the to-be-stimulated target region 20.

As a feasible embodiment, when a distance between the first top segment 1013 and the plane where the to-be-stimulated target region 20 is located is relatively large, the first bottom segment 1011 can stimulate the to-be-stimulated target region 20, and the first rising segment 1012, the first top segment 1013 and the first falling segment 1014 are only used for forming a loop.

As another feasible embodiment, when the distance between the first top segment 1013 and the plane where the to-be-stimulated target region 20 is located is relatively small, the first bottom segment 1011, the first rising segment 1012, the first top segment 1013 and the first falling segment 1014 can all stimulate the to-be-stimulated target region 20.

With continued reference to FIG. 2, both the first rising segment 1012 and the first falling segment 1014 are perpendicular to the plane where the to-be-stimulated target region 20 is located, and along a direction perpendicular to the to-be-stimulated target region 20, the first rising segment 1012 and the first falling segment 1014 have the same dimension from the plane where the to-be-stimulated target region 20 is located.

For example, both the first rising segment 1012 and the first falling segment 1014 are perpendicular to the plane where the to-be-stimulated target region 20 is located, thereby ensuring that the plane where the stereoscopic unit 101 is located is perpendicular to the plane where the to-be-stimulated target region 20 is located. On the one hand, the stimulation effect on the to-be-stimulated target region 20 can be improved. On the other hand, space occupied by the transcranial magnetic stimulation coil 10 can be saved. Along the direction perpendicular to the to-be-stimulated target region 20, the first rising segment 1012 and the first falling segment 1014 have the same dimension from the plane where the to-be-stimulated target region 20 is located. On the one hand, it can be ensured that the stereoscopic unit 101 is a regularly shaped stereoscopic strip. On the other hand, consumables can be reduced.

With continued reference to FIGS. 1 and 2, along the direction perpendicular to the plane where the to-be-stimulated target region 20 is located, a dimension t2 of the first rising segment 1012 is greater than 5 cm.

For example, along the direction perpendicular to the plane where the to-be-stimulated target region 20 is located, the dimension of the first rising segment 1012 is greater than 5 cm so that the distance between the first top segment 1013 and the plane where the to-be-stimulated target region 20 is located can be relatively large, the first bottom segment 1011 can stimulate the to-be-stimulated target region 20, and the first rising segment 1012, the first top segment 1013 and the first falling segment 1014 are only used for forming the loop, thereby avoiding unnecessary stimulation to a region beyond the to-be-stimulated target region 20.

FIG. 4 is a structure diagram of another stereoscopic unit 101 according to an embodiment of the present application. As shown in FIGS. 1 to 4, the first top segment 1013 is a line segment or an endpoint, and a dimension dl of an orthographic projection of the first bottom segment 1011 within the to-be-stimulated target region 20 is greater than a dimension d2 of an orthographic projection of the first top segment 1013 within the to-be-stimulated target region 20.

For example, as a feasible embodiment, referring to FIGS. 1 and 2, since an effect region of magnetic stimulation is mainly determined by a projection of the transcranial magnetic stimulation coil 10 within the to-be-stimulated target region 20, when the first top segment 1013 is the line segment and the dimension dl of the orthographic projection of the first bottom segment 1011 within the to-be-stimulated target region 20 is greater than the dimension d2 of the orthographic projection of the first top segment 1013 within the to-be-stimulated target region 20, it can be ensured that the first bottom segment 1011 can perform stimulation function on the to-be-stimulated target region 20 with a large stimulation range when the first rising segment 1012 has a relatively large dimension from the plane where the to-be-stimulated target region 20 is located.

As another feasible embodiment, referring to FIGS. 1 and 4, when the first top segment 1013 is the endpoint and the dimension d1 of the orthographic projection of the first bottom segment 1011 within the to-be-stimulated target region 20 is greater than the dimension d2 of the orthographic projection of the first top segment 1013 within the to-be-stimulated target region 20, it can be ensured that the first bottom segment 1011 can perform stimulation function on the to-be-stimulated target region 20 with a large stimulation range when the first rising segment 1012 has a relatively large dimension from the plane where the to-be-stimulated target region 20 is located.

In one or more embodiments, the first bottom segment 1011, the first rising segment 1012, the first top segment 1013 and the first falling segment 1014 are integrally disposed.

For example, the first bottom segment 1011, the first rising segment 1012, the first top segment 1013 and the first falling segment 1014 are integrally disposed, thereby ensuring the integrity of each stereoscopic unit 101. On the one hand, a preparation process flow of the transcranial magnetic stimulation coil 10 can be simplified. On the other hand, a good stimulation effect of the coil can be ensured.

In one or more embodiments, for any one of the n stereoscopic units 101 on the side of the transcranial magnetic stimulation coil 10 facing the edge of the to-be-stimulated target region 20, the projection shape of the stereoscopic unit 101 within the to-be-stimulated target region 20 coincides with the edge of the to-be-stimulated target region 20.

For example, for any one of the n stereoscopic units 101 on the side of the transcranial magnetic stimulation coil 10 facing the edge of the to-be-stimulated target region 20, the projection shape of the stereoscopic unit 101 within the to-be-stimulated target region 20 coincides with the edge of the to-be-stimulated target region 20. In this manner, it can be ensured that the transcranial magnetic stimulation coil 10 matches with the edge of the brain function target region and has a relatively good magnetic stimulation effect on the edge of the to-be-stimulated target region 20.

With continued reference to FIG. 1, the transcranial magnetic stimulation coil 10 includes at least two transcranial magnetic stimulation sub-coils, any one of the at least two transcranial magnetic stimulation sub-coils includes multiple stereoscopic units 101 among the n stereoscopic units 101, and the minimum distance t3 between any two adjacent transcranial magnetic stimulation sub-coils among the at least two transcranial magnetic stimulation sub-coils is less than a second preset distance. The minimum distance between any two adjacent transcranial magnetic stimulation sub-coils is limited so that the minimum distance between every two adjacent transcranial magnetic stimulation sub-coils is limited within a certain distance range and the transcranial magnetic stimulation sub-coils can be uniformly arranged within the to-be-stimulated target region 20. In this manner, it can be ensured that the transcranial magnetic stimulation sub-coils perform the stimulation uniformly on the to-be-stimulated target region 20.

In one or more embodiments, the second preset distance is greater than the first preset distance.

For example, the minimum distance t3 between any two adjacent transcranial magnetic stimulation sub-coils may be understood as the minimum linear distance between two adjacent transcranial magnetic stimulation sub-coils, and the second preset distance may be understood as a fixed distance value between two adjacent transcranial magnetic stimulation sub-coils. For example, the second preset distance may be 3 cm or 5 cm, and the second preset distance is greater than the first preset distance. The minimum distance between any two adjacent transcranial magnetic stimulation sub-coils is limited. The minimum distance between every two adjacent transcranial magnetic stimulation sub-coils is limited within a certain distance range so that the transcranial magnetic stimulation sub-coils can be uniformly arranged within the to-be-stimulated target region 20. In this manner, it can be ensured that the transcranial magnetic stimulation sub-coils perform the stimulation uniformly on the to-be-stimulated target region 20.

In an embodiment, for any one of the stereoscopic units 101 on the side of the transcranial magnetic stimulation coil 10 facing the edge of the to-be-stimulated target region 20, the minimum distance t1 between the projection shape within the to-be-stimulated target region 20 and the edge of the to-be-stimulated target region 20 is less than the minimum distance t3 between any two adjacent transcranial magnetic stimulation sub-coils. It may be understood that the minimum distances t1 and t3 are limited so that the transcranial magnetic stimulation sub-coils can also be uniformly arranged within the to-be-stimulated target region 20, thereby ensuring that the transcranial magnetic stimulation sub-coils can perform the stimulation uniformly on the to-be-stimulated target region 20.

In an embodiment, the minimum distance t3 between any two adjacent transcranial magnetic stimulation sub-coils may be a fixed distance value. For example, the minimum distance t3 between any adjacent transcranial magnetic stimulation sub-coils is 3 cm. In this manner, the transcranial magnetic stimulation sub-coils can be better and uniformly arranged within the to-be-stimulated target region 20 to ensure that the transcranial magnetic stimulation sub-coils can perform the stimulation uniformly on the to-be-stimulated target region 20.

FIG. 5 is a structure diagram of an n^{th} stereoscopic unit 101 according to an embodiment of the present application. As shown in FIG. 5, the n^{th} stereoscopic unit 101 among the n stereoscopic units 101 includes a second bottom segment 30 and a second rising segment 40 that are connected in sequence, an end of the second bottom segment 30 includes the start point 11, and an end of the second rising segment 40 includes the end point 12.

For example, the n^{th} stereoscopic unit 101 includes the second bottom segment 30 and the second rising segment 40 that are connected in sequence, that is, the start point 11 of the second bottom segment 30 in the n^{th} stereoscopic unit 101 is connected to the end point 12 of an (n−1)^{th} stereoscopic unit 101, and the end point 12 of the second rising section 40 in the n^{th} stereoscopic unit 101 is connected to an outlet in a housing 50. In this manner, a coil wire can be saved.

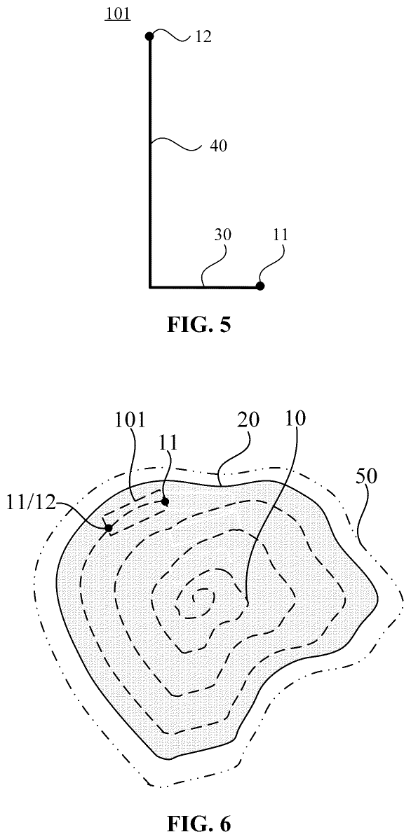

FIG. 6 is a schematic diagram of a projection of another transcranial magnetic stimulation coil 10 within a to-be-stimulated target region 20 according to an embodiment of the present application. As shown in FIG. 6, the transcranial magnetic stimulation coil 10 further includes a housing 50. Along the direction perpendicular to the plane where the to-be-stimulated target region 20 is located, a shape of a surface of the housing 50 on a side of the housing 50 facing the to-be-stimulated target region 20 is the same as the shape of the to-be-stimulated target region 20, the housing 50 surrounds the to-be-stimulated target region 20, and a dimension of the housing 50 is the same as a dimension of multiple stereoscopic units 101. The housing 50 includes an inlet and an outlet for connecting a magnetic stimulation device. The start point 11 of the first stereoscopic unit 101 among the n stereoscopic units 101 is connected to the inlet via a wire, and the end point 12 of the n^{th} stereoscopic unit 101 among the n stereoscopic units 101 is connected to the outlet via a wire.

For example, the housing 50 can protect the transcranial magnetic stimulation coil 10, and the shape of the surface of the housing 50 on the side of the housing 50 facing the to-be-stimulated target region 20 is the same as the shape of the edge of the to-be-stimulated target region 20. That is, the shape of the housing 50 is the same as the shape of the edge of the to-be-stimulated target region 20, and the bottom surface of the housing 50 is formed by expanding the to-be-stimulated target region 20 outward. In addition, the housing 50 surrounds the to-be-stimulated target region 20, that is, the contour of the housing 50 is greater than the contour of the edge of the to-be-stimulated target region 20. On the one hand, the to-be-stimulated target region 20 can be surrounded, thereby avoiding unnecessary stimulation to the region beyond the to-be-stimulated target region 20. On the other hand, it is convenient for a user to wear the transcranial magnetic stimulation coil 10. Along the direction perpendicular to the plane where the to-be-stimulated target region 20 is located, the dimension of the housing 50 is the same as the dimension of the multiple stereoscopic units 101, that is, the housing 50 may be a cylinder with the same height as the multiple stereoscopic units 101.

In an embodiment, the housing 50 includes the inlet and the outlet for connecting the magnetic stimulation device. For example, the wire inlet may be disposed at a position where a side surface of the cylinder housing 50 close to the bottom of the cylinder housing 50, and the wire outlet may be disposed at a position of the top of the housing 50. The coil wire passes through the inlet and the outlet of the housing 50 to form a strip-type vertical coil with a housing, and the shape of the coil matches with the shape of the to-be-stimulated target region 20. The start point 11 of the first stereoscopic unit 101 among the n stereoscopic units 101 is connected to the inlet via the wire, and the end point 12 of the n^{th} stereoscopic unit 101 among the n stereoscopic units 101 is connected to the outlet via the wire. In this manner, the magnetic stimulation device can be connected, thereby achieving the magnetic stimulation effect on the to-be-stimulated target region 20.

In conclusion, the embodiments of the present application provide the transcranial magnetic stimulation coil 10. The transcranial magnetic stimulation coil 10 formed by the multiple stereoscopic units 101 can be arranged in series in the to-be-stimulated target region 20, that is, the local stimulation or the overall stimulation of the to-be-stimulated target region 20 can be achieved. Each stereoscopic unit 101 can generate a corresponding stimulation point, and the stimulation points of the multiple stereoscopic units 101 form the lattice that contributes to the generation of the irregularly shaped stimulation region. That is, the stimulation region matching the brain function target region can be achieved. The formed stimulation lattice depends on the arrangement manner of each stereoscopic unit 101, thereby facilitating the generation of a personalized stimulation region. It may be understood that the personalized stimulation region does not have to correspond to a particular brain function region and also does not have to be a closed region. Stimulation of a connection line or a neural circuit in a neural network can also be customized with the mode of the stereoscopic unit 101.

1. A transcranial magnetic stimulation coil, comprising:
n stereoscopic units, wherein a plane where each of the n stereoscopic units is located intersects a plane where a to-be-stimulated target region is located;
each of the n stereoscopic units comprises a start point and an end point, and the end point of an i^{th} stereoscopic unit among the n stereoscopic units is connected to the start point of an (i+1)^{th} stereoscopic unit among the n stereoscopic units, wherein 1≤i≤n−1), and both i and n are positive integers; and
the transcranial magnetic stimulation coil is arranged in a spiral shape, and for any one of the n stereoscopic units on a side of the transcranial magnetic stimulation coil adjacent to an edge of the to-be-stimulated target region, a minimum distance between a projection shape of the one stereoscopic unit within the to-be-stimulated target region and the edge of the to-be-stimulated target region is less than a first preset distance.

2. The transcranial magnetic stimulation coil according to claim 1, wherein:
at least some of the n stereoscopic units comprise a first bottom segment, a first rising segment, a first top segment and a first falling segment that are connected in sequence; and
an end of the first bottom segment comprises the start point, and an end of the first falling segment comprises the end point.

3. The transcranial magnetic stimulation coil according to claim 2, wherein both the first rising segment and the first falling segment are perpendicular to the plane where the to-be-stimulated target region is located, and along a direction perpendicular to the plane where the to-be-stimulated target region is located, the first rising segment and the first falling segment have a same dimension from the plane where the to-be-stimulated target region is located.

4. The transcranial magnetic stimulation coil according to claim 3, wherein along the direction perpendicular to the plane where the to-be-stimulated target region is located, the dimension of the first rising segment from the plane where the to-be-stimulated target region is located is greater than 5 cm.

5. The transcranial magnetic stimulation coil according to claim 2, wherein the first top segment is a line segment or an endpoint, and a dimension of an orthographic projection of the first bottom segment within the to-be-stimulated target region is greater than a dimension of an orthographic projection of the first top segment within the to-be-stimulated target region.

6. The transcranial magnetic stimulation coil according to claim 2, wherein the first bottom segment, the first rising segment, the first top segment and the first falling segment are integrally disposed.

7. The transcranial magnetic stimulation coil according to claim 1, wherein for any one of the n stereoscopic units on the side of the transcranial magnetic stimulation coil adjacent to the edge of the to-be-stimulated target region, the projection shape of the one stereoscopic unit within the to-be-stimulated target region coincides with the edge of the to-be-stimulated target region.

8. The transcranial magnetic stimulation coil according to claim 1, comprising:
at least two transcranial magnetic stimulation sub-coils, wherein any one of the at least two transcranial magnetic stimulation sub-coils comprises a plurality of stereoscopic units among the n stereoscopic units, and a minimum distance between any two adjacent transcranial magnetic stimulation sub-coils among the at least two transcranial magnetic stimulation sub-coils is less than a second preset distance; and
the second preset distance is greater than the first preset distance.

9. The transcranial magnetic stimulation coil according to claim 2, wherein an n^{th} stereoscopic unit among the n stereoscopic units comprises a second bottom segment and a second rising segment that are connected in sequence; and
an end of the second bottom segment comprises the start point, and an end of the second rising segment comprises the end point.

10. The transcranial magnetic stimulation coil according to claim 1, further comprising:
a housing, wherein along a direction perpendicular to the plane where the to-be-stimulated target region is located, a shape of a surface of the housing on a side of the housing adjacent to the to-be-stimulated target region is the same as a shape of the edge of the to-be-stimulated target region, the housing surrounds the to-be-stimulated target region, and the housing is configured to cover the n stereoscopic units;
the housing comprises an inlet and an outlet for connecting a magnetic stimulation device; and
among the n stereoscopic units, the start point of a first stereoscopic unit is connected to the inlet via a wire, and the end point of an n^{th} stereoscopic unit is connected to the outlet via a wire.
